(19) United States
(12) Patent Application Publication
(10) Pub. No.: US 2025/0107943 A1
(43) Pub. Date: Apr. 3, 2025

(54) UTILIZATION OF AN ADAPTABLE LOW POWER SENSOR TO ENABLE SIMULTANEOUS IN-SYSTEM HIGH DYNAMIC RANGE MEASUREMENT OF RESISTANCE AND CAPACITANCE TO ESTIMATE AND INTELLIGENTLY TRANSMIT THE SATURATION OF A SENSOR ENHANCED DISPOSABLE DIAPER

(71) Applicant: Matthew Napier, Cedar City, UT (US)

(72) Inventor: Matthew Napier, Cedar City, UT (US)

(21) Appl. No.: 18/899,914

(22) Filed: Sep. 27, 2024

Related U.S. Application Data
(60) Provisional application No. 63/586,168, filed on Sep. 28, 2023.
(60) Provisional application No. 63/586,177, filed on Sep. 28, 2023.
(60) Provisional application No. 63/586,179, filed on Sep. 28, 2023.

(51) Int. Cl. A61F 13/42

(52) U.S. Cl. CPC A61F 13/42; A61F 2013/424

(57) ABSTRACT

A wet event device for detecting a wetness event within a diaper. The wet event device includes a front-end sensor configured to be placed within a diaper. The front-end sensor measures the resistance within the diaper and measures the capacitance within the diaper. The wet event device also includes a monitor. The monitor can connect to the front-end sensor and determine from the resistance and capacitance measurements within the diaper when a wet event has occurred.

### CROSS-REFERENCE TO RELATED APPLICATIONS

This application claims the benefit of and priority to U.S. Provisional Patent Application Ser. No. 63/586,168 filed on Sep. 28, 2023 which application is incorporated herein by reference in its entirety.

This application is related to co-pending U.S. patent application Ser. No. ______, filed on Sep. 27, 2024 (Attorney Docket No. 10536-11), and entitled, “SIMULTANEOUS RESISTANCE AND CAPACITANCE MEASUREMENT AND INFERENCING ALGORITHM ENABLING REAL TIME AND IMPROVED INCONTINENCE WET SENSING AND DIAPER SATURATION ESTIMATION”, which application is incorporated herein by reference in its entirety (hereinafter “first related application”).

Co-pending application Ser. No. ______ (Attorney Docket No. 10536-11) claims the benefit of and priority to U.S. Provisional patent application Ser. No. 63/586,177 filed on Sep. 28, 2023 which application is incorporated herein by reference in its entirety.

This application is related to co-pending U.S. patent application Ser. No. ______, filed on Sep. 27, 2024 (Attorney Docket No. 10536-12), and entitled, “THE UTILIZATION OF MULTIVARIABLE REAL-TIME & CALIBRATION DATA INFERENCING TO IMPROVE THE PREDICTION OF A DISPOSABLE DIAPER'S SATURATION LEVEL”, which application is incorporated herein by reference in its entirety (hereinafter “first related application”).

Co-pending application Ser. No. ______ (Attorney Docket No. 10536-12) claims the benefit of and priority to U.S. Provisional patent application Ser. No. 63/586,179 filed on Sep. 28, 2023 which application is incorporated herein by reference in its entirety.

### BACKGROUND OF THE INVENTION

The number of incontinent individuals in the world is rising significantly encompassing the aging population, babies/toddlers, and those who are handicapped/disabled. A cost-effective way to estimate the saturation of a disposable diaper will significantly enhance the quality of life for those people with significant incontinence. Quick changes of a diaper, after an incontinent event, will reduce the risk of skin irritation, UTI's and other diaper related ailments. What is needed is a simple and cost-effective sensor placed directly into the diaper which gives the user or a caregiver information about diaper wetness/saturation. The removable sensor attaches via snaps (or other attachment method) which allows quick and easy diaper changes. Our advanced sensing methods enable this cost-effective solution at a price point lower than competitors.

Sensing the saturation of a diaper with one sensor modality makes it very difficult to repeatedly predict the saturation of a diaper. This multi sensing modality software, when married to a multi sensing front-end sensor, enables multiple sensing modalities to be generated, transmitted and received via IOT message and inferred into a diaper saturation estimate.

### BRIEF SUMMARY OF SOME EXAMPLE EMBODIMENTS

This Summary is provided to introduce a selection of concepts in a simplified form that are further described below in the Detailed Description. This Summary is not intended to identify key features or essential characteristics of the claimed subject matter, nor is it intended to be used as an aid in determining the scope of the claimed subject matter.

One example embodiment includes a wet event device for detecting a wetness event within a diaper. The wet event device includes a front-end sensor configured to be placed within a diaper. The front-end sensor is configured to allow a simultaneous estimation of the resistance within the diaper and the capacitance within the diaper. The wet event device also includes a monitor. The monitor can connect to the front-end sensor and determine from the resistance and capacitance measurements within the diaper when a wet event has occurred.

Another example embodiment includes a wet event device for detecting a wetness event within a diaper. The wet event device includes a front-end sensor configured to be placed within a diaper. The front-end sensor includes a first conducting strip and a second conducting strip. The front-end sensor is configured to allow a simultaneous estimation of the resistance within the diaper and the capacitance within the diaper. The wet event device also includes a monitor. The monitor can connect to the front-end sensor and determine from the resistance and capacitance measurements within the diaper when a wet event has occurred. The monitor includes a communications block, where the communications block is configured to communicate the occurrence of a wet event to a user.

Another example embodiment includes a wet event device for detecting a wetness event within a diaper. The wet event device includes a front-end sensor configured to be placed within a diaper. The front-end sensor includes a first conducting strip. The first conducting strip includes a series of resistors and an electrostatic discharge clamp diode. The front-end sensor also includes a second conducting strip where the second conducting strip includes an electrostatic discharge clamp diode. The front-end sensor is configured to allow a simultaneous estimation of the resistance within the diaper and the capacitance within the diaper. The wet event device also includes a monitor. The monitor is configured to detachably connect to the front-end sensor via a step output general purpose input and output array and determine from the resistance and capacitance measurements within the diaper when a wet event has occurred. The monitor includes a microcontroller unit. The microcontroller is configured to send an input into the front-end sensor via the general purpose input and output array and receive an output from the front-end sensor via the general purpose input and output array. The microcontroller is also configured to estimate the resistance within the diaper environment from the received output from the front-end sensor and estimate the capacitance within the diaper environment from the received output from the front-end sensor. The monitor also includes a communications block. The communications block includes Bluetooth connectivity and Wi-Fi connectivity and is configured to communicate the occurrence of a wet event to a user.

These and other objects and features of the present invention will become more fully apparent from the following description and appended claims, or may be learned by the practice of the invention as set forth hereinafter.

### BRIEF DESCRIPTION OF THE DRAWINGS

To further clarify various aspects of some example embodiments of the present invention, a more particular description of the invention will be rendered by reference to specific embodiments thereof which are illustrated in the appended drawings. It is appreciated that these drawings depict only illustrated embodiments of the invention and are therefore not to be considered limiting of its scope. The invention will be described and explained with additional specificity and detail through the use of the accompanying drawings in which:

FIG. 1 shows an example of a wet event device which detects changes in resistance and capacitance in a diaper; and

FIG. 2 illustrates an example of a front-end sensor within a diaper.

### DETAILED DESCRIPTION OF SOME EXAMPLE EMBODIMENTS

Reference will now be made to the figures wherein like structures will be provided with like reference designations. It is understood that the figures are diagrammatic and schematic representations of some embodiments of the invention, and are not limiting of the present invention, nor are they necessarily drawn to scale.

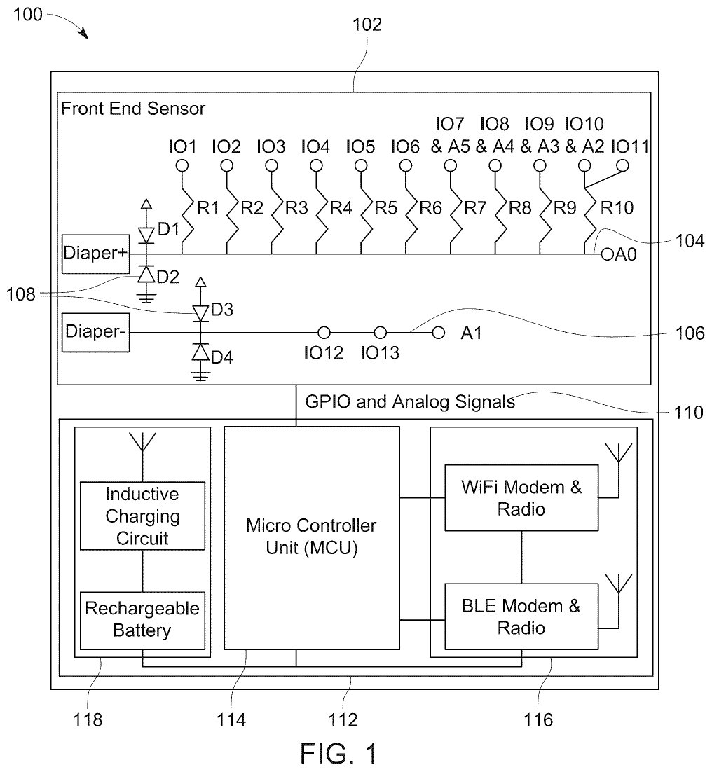

FIG. 1 shows an example of a wet event device 100. The wet event device is capable of detecting a wet event within a diaper. A wet event is any event that can create a sudden change in moisture levels within a diaper environment, such as urination or defecation by the user. Some wet events do not require a change of the diaper. For example, excessive sweating by the user or a small amount of urine may not require a diaper change. Therefore, saturation level is also important to determine. The wet event device 100 detects changes in resistance and capacitance in a diaper, which allows the wet event device 100 to detect a wet event much better than existing sensors. Sensors can be simple when measuring wetness outside of a diaper environment. However, the diaper environment, when placed on a user, is a very dynamic meteorological and electrical environment. For example, wetness can be added to the diaper environment because of moisture from the user's body (e.g., sweat). Therefore, a wet event device 100 must have the ability to both survive this environment and have the capability to accurately and repeatedly measure this environment to estimate the diapers wetness/saturation level while in use.

FIG. 1 shows that the wet event device 100 can include a front-end sensor 102. The front-end sensor 102 is placed within the diaper either as an add-on or built into the diaper. In particular, the front-end sensor 102 is placed within the diaper environment. The front-end sensor 102 can be built into the diaper and sold as a single unit or can be sold as a separate device which is placed within a diaper by a user.

FIG. 1 also shows that the front-end sensor 102 can include a first conducting strip 104. The first conducting strip 104 runs the length of the diaper (front to back). The first conducting strip 104 includes a pair of diodes and a series of resistors connected to input/outputs.

FIG. 1 further shows that the front-end sensor 102 can include a second conducting strip 106. The second conducting strip 106 runs the length of the diaper (front to back). The second conducting strip 106 includes a pair of diodes and a series of inputs/outputs.

The combination of the first conducting strip 104 and the second conducting strip 106 allows for the estimation of the resistance within the diaper environment. This allows the wet event device 100 to determine when resistance within the diaper environment has changed. Moisture within the diaper environment, especially sudden changes in moisture, changes the resistance. However, a change in resistance by itself doesn't indicate a wet event because other changes in the environment can cause a change in resistance. For example, if the user is sweating and the sweat runs down the back of the diaper and interacts with the front-end sensor 102, the resistance measurement would be similar to the resistance change due to a wet event. However, this would not require a diaper change. Therefore, changes in resistance as measured by the first conducting strip 104 can indicate a wet event but are not by themselves definitive.

Likewise, the combination of the first conducting strip 104 and the second conducting strip 106 allows for an estimate of the capacitance within the diaper environment. This allows the wet even device 100 to determine when capacitance within the diaper environment has changed. Moisture within the diaper environment, especially sudden changes in moisture, changes the capacitance. However, a change in capacitance by itself doesn't indicate a wet event because other changes in the environment can cause a change in capacitance. For example, in the sweat example above, the sweat would create a combination of low resistance and low capacitance whereas a wet event would create low resistance and high capacitance. Further, someone standing in a wet diaper would lead to a different capacitance measurement than that same person sitting in that same diaper. Therefore, changes in capacitance as measured by the second conducting strip 106 can indicate a wet event but are not by themselves definitive.

FIG. 1 additionally shows that the front-end sensor 102 can include electrostatic discharge (ESD) clamp diodes 108 on the first conducting strip 104 and the second conducting strip 106. The ESD clamp diodes 108 protect the first conducting strip 104 and the second conducting strip 106 from the harsh environment of the diaper. In particular, the ESD clamp diodes 108 prevent stray electrical signals (e.g., from the user's body, or from static electricity from rubbing against clothing or the user's body) from creating false signals and damage to the first conducting strip 104 and the second conducting strip 106. In some cases, the Diaper Minus connection is tied directly to sensor ground which removes the need for the ESD clamp diodes 108. Sensor ground is a common reference point for all electronics on the sensor and is not to be confused with earth ground.

The first conducting strip 104 and the second conducting strip 106 can create a multi-component pull up resistance circuit which enables: (1) a high dynamic range diaper resistance measurement estimate, (2) the in-wet event device 100 step response parallel Thevenin equivalent resistance estimation and (3) a high dynamic range capacitance measurement estimate. A high dynamic range pull up resistance circuit enables high dynamic range capacitance measurement estimates in the presence of parallel diaper resistance. High dynamic range measurement of both the resistance and the capacitance allows the inference of the diaper over a wide range of states, from disconnected through fully saturated.

FIG. 1 further shows that the wet event device 100 can include a step output general purpose input and output (GPIO) array 110. A GPIO driven voltage into the diaper enables the sensing of the diaper's resistance capacitance (RC) time constant waveform. The step output GPIO array 110 can take the form of a plug or connector which allows a front-end sensor 102 to be connected to a monitor 112.

FIG. 1 additionally shows that the wet event device 100 can include a monitor 112. The monitor 112 produces an input signal which is sent to the front-end sensor 102 via the step output GPIO array 110 and receives an output signal from the front-end sensor 102 via the step output GPIO array 110. The output signal allows the controller to determine measurements and from those measurements estimate the resistance and the capacitance.

FIG. 1 moreover shows that the wet event device 102 can include a micro-controller unit (“MCU”) 114. An MCU 114 is a small computer on a single integrated circuit. An MCU 114 contains one or more CPUs (processor cores) along with memory and programmable input/output peripherals. Program memory may also be included. Alternatives to an MCU 114 are FPGAs, DSPs, ASICs or any other equivalent device. MCUs 114 are designed for embedded applications, in contrast to the microprocessors used in personal computers or other general-purpose applications consisting of various discrete chips. Thus, the MCU 114 can use the known input into the front-end sensor 102 and the received output from the front-end sensor 102 to measure the resistance and estimate the Thevenin resistance and capacitance. Configuration of the front-end sensor 102 and step output GPIO array 110 during periods of non-sensing is critical to providing a more stable and grounded/common environment within the diaper and the attached front-end sensor 102. This is enabled by driving both diaper inputs to ground by the MCU 114 via the GPIO array 110 connected to the front-end sensor 102.

Utilizing the derived Thevenin resistance and measured time constant the diapers capacitance is estimated by the MCU 114. Resistance pull-up strength as measured by the front-end sensor 102 is critical to the capacitance estimate. As the diaper becomes more saturated the distributive resistance value of the diaper drops. I.e., resistance measurements begin to differ in various areas of the diaper environment. Employing a high dynamic range pull up resistance circuit on the first conducting strip 104 enables the ability to accurately measure the changing resistance within the diaper environment but also enables a data set which allows for an accurate estimate of the diaper's capacitance.

Critical to the repeatability and accuracy of the capacitance estimate measurement in this multi-mode diaper sensing environment is the requirement for the pull up resistance to track the resistance of the diaper and the pull up value selected be as close as possible to the current diaper resistance as controlled by the MCU 114. Another measure of the diaper's capacitance estimate accuracy is the shape of the rise/fall RC waveform and the resulting time constant, also determined by the MCU. The slew rate of this RC waveform directly correlates to the pull up resistor selected and the current diaper resistance.

Very important to this measurement is optimizing this waveform's slew rate from the MCU 114 to be as fast as possible while also being long enough to get adequate samples. If the resistance chosen is too high (weak) the RC time constant becomes very long and the slope is very shallow which adds uncertainty (noise) into determining when the time constant of the diaper has been met. If the pull up is too low (strong) the RC time constant is very fast, and the ADC circuit bandwidth is not sufficient to collect enough samples to enable an accurate capacitance estimate. The number of analog to digital converter (ADC) samples captured between the time constant start voltage and end voltage is a first order method to know how accurate and repeatable the capacitance measurement is.

Utilizing this measure, the pull up resistance can be tuned further to enhance the accuracy of the capacitance estimate. Rapid and consistent discharge of the diaper's capacitance is also required to enable repeatable measurement. Utilizing the multi-mode capability of the micro-controller (MCU) GPIO the sensor can rapidly and safely discharge the parasitic capacitance on the diaper by driving the input/output (I/O) connected to the diaper two conductor sensors low for a brief period of time prior to the next measurement. In addition, temperature compensation can be applied to both the resistance and capacitance measurement to improve measurement estimates accuracy over time and changing thermal conditions.

In addition, the MCU 114 controls the sensing within the front-end sensor 102 along with multiple other functions described below. The MCU provides low duty cycle voltage and current biasing to the first conducting strip 104 and the second conducting strip 106 of the front-end sensor 102 and grounds the front-end sensor 102. In addition, the MCU 114 transmits the relevant information about a wet event to a caregiver, as described below.

FIG. 1 also shows that the MCU 114 is connected to a communications block 116. The communication block 116 utilizes dual radio band, BLE and Wi-Fi IOT connectivity to provide reliable and dependable communication to external cloud-based databases, a user and/or a caregiver. When used together the sensors connection reliability is improved, dynamic communication power shaping capability is enabled, and user environment range is significantly extended. I.e., the combination of BLE and wi-fi connectivity is both a power saving measure and reliability measure. If BLE enabled device is available, sending the message via BLE vs wi-fi is a lot less power which equals longer battery life. However, with both BLE and wi-fi available to send the message there is a higher probability of the notification making it to the user. In addition, low power operation is critical to preserve sensor battery life and improve user experience. The wetness/saturation sensor measurement data must be transmitted to the user/caregiver to provide the sensor to human control loop enabling a higher standard of care this wet event device 100 will provide.

FIG. 1 additionally shows that the MCU 114 is powered by a rechargeable battery 118 which can be charged inductively or through a wired connection (or both). This allows for mobility of the user as they are able to move around without being connected to an independent power source, such as a wall socket. In addition, wireless charging allows for the monitor 112 to be water resistant and reliable.

The presence of a front-end sensor 102 and an attachable monitor 112 allows for high sensitivity with low cost. In particular, the front-end sensor 102 should not be used multiple times. First, doing so is not hygienic. Second, the environment within the diaper is harsh and is likely to cause damage to a front-end sensor 102. Therefore, the simple nature of the first conducting strip 104 and the second conducting strip 106 integrated into a diaper allows for a combination of high sensitivity and low cost. In particular, the utilization of a simple two strip conductor within the front-end sensor 102 placed in a disposable diaper enables a mass producible and cost-effective product capable of sensing diaper saturation. In contrast, the monitor 112 is reusable because it is not placed in the harsh diaper environment. The monitor 112 is more expensive but can be used for a long period of time with multiple front-end sensors 102, reducing the cost per use. In addition, critical to accurate sensing of a wetness event is the placement of the conductive elements such that they are fully exposed to the internal environment of the diaper. Therefore, a disposable front-end sensor 102 and reusable monitor 112 creates a balance between sensitivity and cost. In addition, because the front-end sensor 102 runs the length of the diaper, allowing sensing of wet events at any location within the diaper (i.e., if the diaper is placed backward, the wet even sensing would work just as well). Further, having the sensor 102 being able to be placed into the diaper adds only a few cents per diaper, whereas fully integrating a sensor into a diaper would be far more costly. Other systems us an RFID to gather and transmit information but this requires the power source to be near the diaper and makes the user uncomfortable.

The described configuration includes a number of benefits relative to current products. The wet event device 100 utilizes the MCUs 114 low power state in between sensor measurements to preserve power. The step output GPIO array 110 configuration ensures the front-end sensor 102 is not biased and is grounded in between sensor measurements. Sensor measurement data is analyzed to determine if the current measurement needs to be transmitted outside the device.

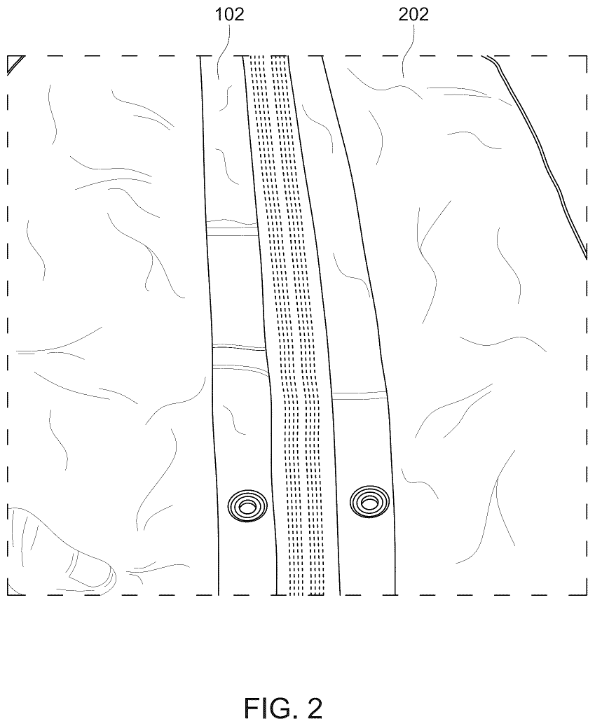

FIG. 2 illustrates an example of a front-end sensor 102 within a diaper 202. The front-end sensor 102 can be integrated within the diaper 202 or can be placed within the diaper 202 after purchase. I.e., the front-end sensor 102 can be placed within a sensorless diaper 202 or other devices such as a pad.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A wet event device for detecting a wetness event within a diaper, the wet event device comprising:
a front-end sensor configured to be placed within a diaper, wherein the front-end sensor is configured to allow a simultaneous estimation of:
the resistance within the diaper; and
the capacitance within the diaper; and
a monitor, wherein the monitor:
can connect to the front-end sensor; and
determine from the resistance and capacitance measurements within the diaper when a wet event has occurred.

2. The wet event device of claim 1, wherein the monitor alerts a user when a wet event has occurred.

3. The wet event device of claim 1, wherein the connection between the front-end sensor and the monitor is detachable.

4. The wet event device of claim 1, wherein the connection between the front-end sensor and the monitor includes a general purpose input and output array.

5. A wet event device for detecting a wetness event within a diaper, the wet event device comprising:
a front-end sensor configured to be placed within a diaper, wherein the front-end sensor:
includes a first conducting strip;
includes a second conducting strip; and
is configured to allow a simultaneous estimation of:
the resistance within the diaper; and
the capacitance within the diaper; and
a monitor, wherein the monitor:
is configured to connect to the front-end sensor;
determine from the resistance and capacitance measurements within the diaper when a wet event has occurred; and
includes a communications block, wherein the communications block is configured to communicate the occurrence of a wet event to a user.

6. The wet event device of claim 5 wherein the communications block includes Bluetooth connectivity.

7. The wet event device of claim 5 wherein the communications block includes Wi-Fi connectivity.

8. The wet event device of claim 5 wherein the first conducting strip includes a series of resistors.

9. The wet event device of claim 5, wherein the first conducting strip includes an electrostatic discharge clamp diode.

10. The wet event device of claim 5, wherein the second conducting strip includes an electrostatic discharge clamp diode.

11. The wet event device of claim 5, wherein the monitor includes a microcontroller unit.

12. The wet event device of claim 5, wherein the microcontroller unit sends an input into the front-end sensor and receives an output from the front-end sensor.

13. The wet event device of claim 5, wherein the microcontroller unit estimates the capacitance within the diaper environment from the received output from the front-end sensor.

14. The wet event device of claim 5, wherein the microcontroller unit estimates the resistance within the diaper environment from the received output from the front-end sensor.

15. A wet event device for detecting a wetness event within a diaper, the wet event device comprising:
a front-end sensor configured to be placed within a diaper, wherein the front-end sensor:
includes a first conducting strip, wherein the first conducting strip includes:
a series of resistors; and
an electrostatic discharge clamp diode;
includes a second conducting strip, wherein the second conducting strip includes an electrostatic discharge clamp diode; and
is configured to allow a simultaneous estimation of
the resistance within the diaper; and
the capacitance within the diaper;
a monitor, wherein the monitor:
is configured to detachably connect to the front-end sensor via a general purpose input and output array;
determines from the resistance and capacitance measurements within the diaper when a wet event has occurred;
includes a microcontroller unit, wherein the microcontroller is configured to:
send an input into the front-end sensor via the general purpose input and output array;
receive an output from the front-end sensor via the general purpose input and output array;
estimate the resistance within the diaper environment from the received output from the front-end sensor; and
estimate the capacitance within the diaper environment from the received output from the front-end sensor; and
includes a communications block, wherein the communications block:
includes Bluetooth connectivity;
includes Wi-Fi connectivity; and
is configured to communicate the occurrence of a wet event to a user.

16. The wet event device of claim 15 wherein the front-end sensor can measure the resistance within at least two regions of the diaper environment.

17. The wet event device of claim 15 wherein the first conducting strip and the second conducting strip create a multi-component pull up resistance circuit.

18. The wet event device of claim 15 wherein the monitor includes a battery capable of powering the microcontroller unit.

19. The wet event device of claim 15 wherein the microcontroller drives the front-end sensor to ground via the step output GPIO array before sending the input signal to the front-end sensor.
